SEQUENCE LISTING (14 sequences)

SEQ ID NO: 1 - DNA, 517 bp, Nicotiana tabacum
tctagaaaccatgaagaatttgattttcctaacgatgtttctgactatattactacaaacaaacgccaataatctagtagaaactacatgcaaaaacacaccaaattaccaactttgtctgaaaactctgctttcggacaaacgaagtgcaacaggggatatcacaacgttggcactaattatggtcgatgcaataaaagctaaagctaatcaggctgcagtgacaatttcgaaactccggcattcgaatccccctgcagcttggaaaggtcctttgaaaaactgtgccttttcatataaggtaattttaacagcaagtttgcctgaagcaattgaagcattgacaaaaggagatccaaaatttgctgaagatggaatggtaggttcatctggagatgcacaagaatgtgaggagtatttcaagggtagtaaatcaccattttctgcattaaatatagcagttcatgaactttctgatgttgggagagctattgtcagaaatttattgtgagagctc

SEQ ID NO: 2 - protein, 166 aa, Nicotiana tabacum
MKNLIFLTMFLTILLQTNANNLVETTCKNTPNYQLCLKTLLSDKRSATGDITTLALIMVDAIKAKANQAAVTISKLRHSNPPAAWKGPLKNCAFSYKVILTASLPEAIEALTKGDPKFAEDGMVGSSGDAQECEEYFKGSKSPFSALNIAVHELSDVGRAIVRNLL

SEQ ID NO: 3 - DNA, 535 bp, Nicotiana tabacum
tctagaaaccatgagaaacttattccccatatttatgttaatcaccaatctagcattcaacgacaacaacaacagtaataatatcatcaacacgacctgcagagccaccacaaactaccccttgtgcctcaccaccctccactctgatccccgtacctccgaggccgagggggcggacctcaccaccctcggcctcgtcatggtagatgcggtaaaattaaagtccatcgaaataatgaaaagtataaaaaaactcgaaaaatcgaaccccgagttgagactacctcttagccaatgttacatagtgtattatgctgttctacatgctgatgtaactgttgctgttgaagctttaaaaagaggagtccctaaatttgctgaaaatggaatggttgatgttgctgtagaagcagaaacttgtgagtttagttttaagtataatggattggtttctccagtttctgatatgaataaggagattattgaactgtcttctgtggctaaatctattattagaatgctattatgagagctc

SEQ ID NO: 4 - protein, 172 aa, Nicotiana tabacum
MRNLFPIFMLITNLAFNDNNNSNNIINTTCRATTNYPLCLTTLHSDPRTSEAEGADLTTLGLVMVDAVKLKSIEIMKSIKKLEKSNPELRLPLSQCYIVYYAVLHADVTVAVEALKRGVPKFAENGMVDVAVEAETCEFSFKYNGLVSPVSDMNKEIIELSSVAKSIIRMLL

SEQ ID NO: 5 - DNA, 953 bp, Arabidopsis thaliana
aagcttgcaaaatagcacaccatttatgtttatattttcaaattatttattacatttcaatatttcataagtgtgattttttttttttttgtcaatttcataagtgtgatttgtcatttgtattaaacaattgtatcgcgcagtacaaataaacagtgggagaggtgaaaatgcagttataaaactgtccaataatttactaacacatttaaatatctaaaaagagtgtttcaaaaaaaattcttttgaaataagaaaagtgatagatatttttacgctttcgtctgaaaataaaacaataatagtttattagaaaaatgttatcaccgaaaattattctagtgccacttgctcggatcgaaattcgaaagttatattctttctctttacctaatataaaaatcacaagaaaaatcaatccgaatatatctatcaacatagtatatgcccttacatattgtttctgacttttctctatccgaatttctcgcttcatggtttttttttaacatattctcatttaattttcattactattatataactaaaagatggaaataaaataaagtgtctttgagaatcgaacgtccatatcagtaagatagtttgtgtgaaggtaaaatctaaaagatttaagttccaaaaacagaaaataatatattacgctagaaaagaagaaaataattaaatacaaaacagaaaaaaataatatacgacagacacgtgtcacgaagataccctacgctatagacacagctctgttttctcttttctatgcctcaaggctctcttaacttcactgtctcctcttcggataatcctatccttctcttcctataaatacctctccactcttcctcttcctccaccactacaaccaccgcaacaaccaccaaaaaccctctcaaagaaatttcttttttttcttactttcttggtttgtcaaatctaga

SEQ ID NO: 6 - DNA, 882 bp, Artificial Sequence
  rolC promoter
aagcttaaagttggcccgctattggatttcgcgaaagcggcattggcaaacgtgaagattgctgcattcaagatactttttctattttctggttaagatgtaaagtattgccacaatcatattaattactaacattgtatatgtaatatagtgcggagattatctatgccaaaatgatgtattaataatagcaataataatatgtgttaatctttttcaatcgggaatacgtttaagcgattatcgtgttgaataaattattccaaaaggaaatacatggttttggagaacctgctatagatatatgccaaatttacactagtttagtgggtgcaaaactattatctctgtttctgagtttaataaaaaataaataagcagggcgaatagcagttagcctaagaaggaatggtggccatgtacgtgcttttaagagaccctataataaattgccagctgtgttgctttggtgccgacaggcctaacgtggggtttagcttgacaaagtagcgcctttccgcagcataaataaaggtaggcgggtgcgtcccattattaaaggaaaaagcaaaagctgagattccatagaccacaaaccaccattattggaggacagaacctattccctcacgtgggtcgctagctttaaacctaataagtaaaaacaattaaaagcaggcaggtgtcccttctatattcgcacaacgaggcgacgtggagcatcgacagccgcatccattaattaataaatttgtggacctatacctaactcaaatatttttattatttgctccaatacgctaacagctctggattataaatagtttgaatgcttcgagttatgggtacaagcaacctgtttcctactttgttaactctaga

SEQ ID NO: 7 - DNA, 1004 bp, Artificial Sequence
  rbcS promoter
aagcttagacaaacaccccttgttatacaaagaatttcgctttacaaaatcaaattcgagaaaataatatatgcactaaataagatcattcggatctaatctaaccaattacgatacgctttgggtacacttgatttttgtttcagtggttacatatatcttgttttatatgctatctttaaggatctgcacaaagattatttgttgatgttcttgatggggctcagaagatttgatatgatacactctaatctttaggagataccagccaggattatattcagtaagacaatcaaattttacgtgttcaaactcgttatcttttcattcaaaggatgagccagaatctttatagaatgattgcaatcgagaatatgttcggccgatatgcctttgttggcttcaatattctacatatcacacaagaatcgaccgtattgtaccctctttccataaaggaaaacacaatatgcagatgcttttttcccacatgcagtaacatataggtattcaaaaatggctaaaagaagttggataacaaattgacaactatttccatttctgttatataaatttcacaacacacaaaagcccgtaatcaagagtctgcccatgtacgaaataacttctattatttggtattgggcctaagcccagctcagagtacgtgggggtaccacatataggaaggtaacaaaatactgcaagatagccccataacgtaccagcctctccttaccacgaagagataagatataagacccaccctgccacgtgtcacatcgtcatggtggttaatgataagggattacatccttctatgtttgtggacatgatgcatgtaatgtcatgagccacaagatccaatggccacaggaacgtaagaatgtagatagatttgattttgtccgttagatagcaaacaacattataaaaggtgtgtatcaataggaactaattcactcattggattcatagaagtccattcctcctaagtatctaga

SEQ ID NO: 8 - DNA, 870 bp, Artificial Sequence
  CaMV 35S promoter
aagcttgcatgcctgcaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctag

SEQ ID NO: 9 - DNA, 558 bp, Artificial Sequence
  Artificial phosphinothricin resistance gene
tcgacatgtctccggagaggagaccagttgagattaggccagctacagcagctgatatggccgcggtttgtgatatcgttaaccattacattgagacgtctacagtgaactttaggacagagccacaaacaccacaagagtggattgatgatctagagaggttgcaagatagatacccttggttggttgctgaggttgagggtgttgtggctggtattgcttacgctgggccctggaaggctaggaacgcttacgattggacagttgagagtactgtttacgtgtcacataggcatcaaaggttgggcctaggatccacattgtacacacatttgcttaagtctatggaggcgcaaggttttaagtctgtggttgctgttataggccttccaaacgatccatctgttaggttgcatgaggctttgggatacacagcccggggtacattgcgcgcagctggatacaagcatggtggatggcatgatgttggtttttggcaaagggattttgagttgccagctcctccaaggccagttaggccagttacccagatctgag

SEQ ID NO: 10 - DNA, 30 bp, Artificial Sequence
  AscI-PAT.F primer
gcttggcgcgcccatggagtcaaagattca

SEQ ID NO: 11 - DNA, 29 bp, Artificial Sequence
  Nhel-PAT.R primer
gcttgctagcgagctcggtacccactgga

SEQ ID NO: 12 - protein, 427 aa, Artificial Sequence
  Enoylpyrovyl-shikimate 3 phosphate synthase (EPSPS) gene
MESLTLQPIAHVDGTINLPGSKTVSNRALLLAALAHGKTVLTNLLDSDDVRHMLNALTALGVSYTLSADRTRCEIIGNGGPLHAEGALELFLGNAGTAMRPLAAALCLGSNDIVLTGEPRMKERPIGHLVDALRLGGAKITYLEQENYPPLRLQGGFTGGNVDVDGSVSSQFLTALLMTAPLAPEDTVIRIKGDLVSKPYIDITLNLMKTFGVEIENQHYQQFVVKGGQSYQSPGTYLVEGDASSASYFLAAAAIKGGTVKVTGIGRNSMQGDIRFADVLEKMGATICWGDDYISCTRGELNAIHMDMNHIPDAAMTIATAALFAKGTTRLRNIYNWRVKETDRLFAMATELRKVGAEVEEGHDYIRITPPEKLNFAEIATYNDHRMAMCFSLVALSDTPVTILDPKCTAKTFPDYFEQLARISQAA

SEQ ID NO: 13 - protein, 55 aa, Brassica napus
MAYSMLSSATVVSSPAQAAMVAPFTGLKSSAAFPVTRKTDTDITSMASNGGRVNS

SEQ ID NO: 14 - DNA, 1838 bp, Artificial Sequence
  GLY-R gene
gctagcaaatatttcttgtcaaaaatgctccactgacgttccataaattcccctcggtatccaattagagtctcatattcactctcaatccaaataatctgcaccggatctggatcgtttcgcatggcttattctatgctttcttctgctactgttgtttcttctccagctcaagctgctatggttgctccatttactggacttaagtcttctgctgcttttccagttactagaaagactgatactgatattacttctatggcttctaatggaggaagagttaattctatggaatctcttactcttcaaccaattgctcatgttgatggaactattaatcttccaggatctaagactgtttctaatagggcacttcttcttgctgctcttgctcatggaaagactgttcttactaatcttcttgattctgatgatgttagacacatgcttaatgctcttactgctcttggagtttcttatactctttctgctgatagaactagatgtgaaattattggaaatggaggaccacttcatgctgaaggtgcacttgaactttttcttggaaatgctggaactgctatgagaccacttgctgctgctctttgtcttggatctaatgatattgttcttactggagaaccaagaatgaaggaaagaccaattggacatcttgttgatgctcttagacttggaggagctaagattacttatcttgaacaagaaaattatccaccacttagacttcaaggaggatttactggaggaaatgttgatgttgatggatctgtttcttctcaatttcttactgctcttcttatgactgctccacttgctccagaagatactgttattagaattaagggtgatttagtttctaagccatatattgatattactcttaatcttatgaagacttttggagttgaaattgaaaatcaacattatcaacaatttgttgttaagggaggacaatcttatcaatctccaggaacttatcttgttgaaggagatgcttcttctgcttcttattttcttgctgctgctgctattaagggaggaactgttaaggttactggaattggaagaaattctatgcaaggagatattagatttgctgatgttcttgaaaagatgggagctactatttgttggggagatgattatatttcttgtactagaggagaacttaatgctattcacatggatatgaatcatattccagatgctgctatgactattgctactgctgctctttttgctaagggaactactagacttagaaacatctataattggagagttaaggaaactgatagactttttgctatggctactgaacttagaaaggttggagctgaagttgaagaaggacatgattatattagaattactccaccagaaaagttgaattttgctgaaattgctacttacaacgaccatagaatggctatgtgtttttctcttgttgctctttctgatactccagttactattcttgatccaaagtgtactgctaagacttttccagattattttgaacaacttgctagaatttctcaagctgcttgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggcgcgcc

(19) United States
(12) Patent Application Publication
(10) Pub. No.: US 2017/0096686 A1
(43) Pub. Date: Apr. 6, 2017

(54) TRANSGENIC TOBACCO PLANTS FOR ENHANCED BIOETHANOL PRODUCTION

(71) Applicant: Tyton BioSciences, LLC, Danville, VA (US)

(72) Inventors: Vyacheslav Adrianov, Doylestown, PA (US); Igor Kostenyuk, Winter Haven, FL (US); Peter Majeranowski, Satellite Beach, FL (US); Iulian Bobe, Danville, VA (US); Mintu K. Desai, Hillsborough, NC (US)

(21) Appl. No.: 15/314,191

(22) PCT Filed: May 27, 2015
(86) PCT No.: PCT/US2015/032743

§ 371 Date: Nov. 28, 2016

Related U.S. Application Data
(60) Provisional application No. 62/004,181, filed on May 28, 2014.

(51) Int. Cl. C12P 7/06; C07K 14/415; C12N 9/10; C12N 15/82

(52) U.S. Cl. CPC C12P 7/06; C12N 15/8245; C12N 15/8226; C12N 15/8227; C07K 14/415; C12N 9/1092; C12Y 205/01019; C12N 15/8275

(57) ABSTRACT

Genetically modified tobacco plants are provided having altered hexose accumulation. Methods are provided for producing ethanol from fermentation of tobacco biomass derived from the tobacco plants having altered hexose accumulation. The altered hexose accumulation can be an increase in total hexose content or an increase in hexose content in the phloem or the roots/shoots as compared to non-genetically modified tobacco plants. Expression vectors are provided for tobacco plant transformation having a gene encoding a sucrose invertase inhibitor operably linked to a promoter, such that expression of the inhibitor in the plant can increase and/or alter hexose accumulation in the plant. The genetically modified tobacco plants having altered hexose accumulation can further contain a transgenic construct to confer resistance to a glyphosate herbicide or a phosphinothricin (PPT) herbicide.

### CROSS REFERENCE TO RELATED APPLICATIONS

This application claims the benefit of U.S. provisional patent application No. 62/004,181 filed May 28, 2014, the disclosure of which is hereby incorporated by reference in its entirety.

### TECHNICAL FIELD

The present subject matter relates to tobacco plants having a transgenic expression construct to confer altered hexose accumulation in the plant. Transgenic plants having both the transgenic expression construct to confer altered hexose accumulation and a transgene that confers herbicide tolerance to the plant are also provided. Biomass from the transgenic tobacco plants including juice derived from the biomass of the transgenic tobacco plants can be useful for producing bioethanol.

### BACKGROUND

Renewable energy from biomass has the potential to reduce dependency on fossil fuels and its negative environmental impact. Realization of this potential will require the development of high yielding biomass production systems. Recent achievements in genomic research provide an excellent basis for translation of genomics information into plant cultivar improvement. New opportunities emerge to develop the technological foundation for an environmentally sustainable biological production of plant biomass that can be economically converted into bioethanol and biodiesel. Despite recent progress with utilization of such “energy” plants as sugar cane, corn and algae as a renewable feedstock for bioenergy production, introduction of additional crop plants is still required due to diversity of agronomical requirements in different regions.

As an example, tobacco plants were recently shown to be able to accumulate elevated amounts of fatty acids, doubling the amount of bio-diesel oil that can be extracted from leaf and stem biomass as a result of genetic modification (Andrianov et al., 2010). The major advantage for utilizing tobacco as an energy biomass feedstock is that it is a well established non-food industrial crop that is cultivated in more than 100 countries around the world with a solid record of genetic modification. When grown for energy production rather than smoking, tobacco biomass can be generated more efficiently and inexpensively than almost any other agricultural crop. In addition, tobacco can be grown on land not involved in food production, such that its production for energy biomass is not replacing growth of a food crop. Further, use of tobacco as a renewable resource as energy biomass promotes energy independence.

As a biomass for cellulosic ethanol fermentation, tobacco has two main advantages over existing feedstocks: a high amount of easily fermentable sugars, and a low content of lignin, which in other lignocellulosic feedstock significantly hampers the fermentation process and contributes to their high costs. Tobacco biomass is naturally rich in sugars and starch and low-lignin cellulose. While there is wide variation among tobacco types, generally tobacco contains 15-20% sugars, 8-14% starch and 30-40% cellulose per dry weight.

However, and in contrast to the kernel of corn plants, a major disadvantage to tobacco for ethanol fermentation is that the sugar in tobacco is not largely localized in a tissue that can be easily fermented to produce ethanol. In addition, the amount of free sugars in plant tissues that can be directly fermented into ethanol is under tight physiological control. The sucrose-cleaving enzymes of plants have multiple functions that directly or indirectly affect different processes. According to their pH optima, plant invertases can be divided into two categories: neutral and acid invertases. Acid invertases are able to cleave sucrose in extracellular compartments such as the vacuole (vacuolar invertase; VI) or the apoplastic space (cell wall invertase; CWI). The activity of acid invertases is inhibited by small polypeptides termed Sucrose Invertase Inhibitors (SIS) specific for a particular invertase type. Ectopic expression of sucrose invertase inhibitors, even when performed under constitutive promoter regulation, caused dramatic changes in hexose accumulation during plant development (Greiner et al., 1999). An additional disadvantage is that tobacco biomass contains levels of nicotine that can be toxic to certain organisms such as the microbial strains used in the fermentation reaction.

The methods of biotechnology have been applied to tobacco for improvement of the agronomic traits and the quality of the product. One such agronomic trait is herbicide tolerance. Different broad spectrum herbicides, such as 2,4-dichlorophenoxyacetic acid (2,4-D), bromoxynil, glufosinate and glyphosate are used in plant biotechnology. Among others glyphosate is currently considered as an ideal herbicide to use in resistant crops due to its broad spectrum, nonselective weed control, favorable safety profile, low mammalian toxicity, benign environmental impact favor, relatively low cost and familiarity with growers that can be a great advantage for agricultural technique. Glyphosate effectively blocks the biosynthesis of aromatic amino acids by irreversibly inhibiting activity of the 5-enolpyruvyl shikimate-3-phosphate synthase (EPSP synthase) enzyme [EC 2.5.1.19]. In plant cells the EPSP synthase is a chloroplast-localized enzyme which is encoded by the nuclear genome.

Thus, while the cost of enzymes used for fermentation is decreasing and there has been progress in the optimization of biomass pretreatment methods and development of more efficient microbial strains, multiple hurdles remain before tobacco biomass can be used economically as a feedstock for the production of biofuel ethanol. Thus, an unmet need remains for improved tobacco biomass that can be used for production of ethanol from fermentation of tobacco biomass. The present disclosure provides compositions and methods that enable enhanced production of ethanol from fermentation of tobacco biomass.

### SUMMARY

In one embodiment, the presently disclosed subject matter provides a genetically modified tobacco plant having altered hexose accumulation, the plant comprising a gene encoding a sucrose invertase inhibitor operably linked to a promoter, such that the hexose accumulation in the modified plant is one or both of increased and altered as compared to a non-genetically modified tobacco plant.

In one embodiment, the presently disclosed subject matter provides a seed of a genetically modified tobacco plant having altered hexose accumulation, the plant comprising a gene encoding a sucrose invertase inhibitor operably linked to a promoter, such that the hexose accumulation in the modified plant is one or both of increased and altered as compared to a non-genetically modified tobacco plant.

In one embodiment, the presently disclosed subject matter provides a vector for transformation of a tobacco plant, the vector comprising a gene encoding a sucrose invertase inhibitor operably linked to a promoter, such that expression of the sucrose invertase inhibitor in the tobacco plant one of increases and alters hexose accumulation in the plant.

In one embodiment, the presently disclosed subject matter provides a method for producing ethanol from tobacco biomass, the method comprising fermenting a tobacco biomass extract such that ethanol is produced from the fermentation, wherein the tobacco biomass extract is derived from a genetically modified tobacco plant having altered hexose accumulation, the plant comprising a gene encoding a sucrose invertase inhibitor operably linked to a promoter, such that the hexose accumulation in the modified plant is one or both of increased and altered as compared to a non-genetically modified tobacco plant.

### BRIEF DESCRIPTION OF THE FIGURES

FIG. 1 is a PCR gel showing the amplification of NPT-II in various samples from tobacco transgenic lines having a pBI_SUC2-Nt_inh1 or a pBI_SUC2-Nt_inhh vector construct: S76, S83, TN81, TN85, NC85*, S83, NC87, K811, K813 and S79, and wild-type (untransformed) lines K326 and SHIREY LC, according to one or more embodiments of the present disclosure. One of the plasmid constructs containing the NPT-II gene (pBI_SUC2-Nt_inh1 or pBI_SUC2-Nt_inhh) was used as a positive control (+ve Control).

FIGS. 2A-2C are histograms showing the sugar levels in untransformed tobacco (NC WT) compared to levels in transgenic tobacco line NC87 which is a NC567 tobacco variety containing a pBI_SUC2-Nt_inhh vector construct according to one or more embodiments of the present disclosure. A) Histogram showing the percent free hexose (Free Sugar) measured in the tobacco juice extracted from each of the transgenic (NC87) and wild-type (NC WT) lines; B) Histogram graph showing the total hexose (Total Sugar) as measured by percent of wet weight of the biomass extracted from each of the transgenic (NC87) and wild-type (NC WT) lines; and C) Histogram graph showing the total hexose (Total Sugar) as measured by percent of dry weight of the biomass extracted from each of the transgenic (NC87) and wild-type (NC WT) lines.

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

Unless otherwise defined, all technical terms used herein have the same meaning as commonly understood by one of ordinary skill in the art to which this disclosure belongs.

The term “gene” as used herein refers to an element or combination of elements that are capable of being expressed in a cell, either alone or in combination with other elements. In general, a gene comprises (from the 5′ to the 3′ end): (1) a promoter region, which includes a 5′ non-translated leader sequence capable of functioning in plant cells; (2) a structural gene or polynucleotide sequence, which codes for the desired protein; and (3) a 3′ non-translated region, which typically causes the termination of transcription and the polyadenylation of the 3′ region of the RNA sequence. Each of these elements is operably linked by sequential attachment to the adjacent element. A gene comprising the above elements is inserted by standard recombinant DNA methods into a plant expression vector.

As used herein “promoter” refers to a region of a DNA sequence active in the initiation and regulation of the expression of a structural gene. This sequence of DNA, usually upstream to the coding sequence of a structural gene, controls the expression of the coding region by providing the recognition for RNA polymerase and/or other elements required for transcription to start at the correct site.

As used herein, “polynucleotide” includes cDNA, RNA, DNA/RNA hybrid, anti-sense RNA, ribozyme, genomic DNA, synthetic forms, and mixed polymers, both sense and antisense strands, and may be chemically or biochemically modified to contain non--natural or derivatized, synthetic, or semi-synthetic nucleotide bases. Also, included within the scope of the invention are alterations of a wild type or synthetic gene, including but not limited to deletion, insertion, substitution of one or more nucleotides, or fusion to other polynucleotide sequences, provided that such changes in the primary sequence of the gene do not substantially alter the expressed polypeptide's activity.

As used herein, “polypeptide” is used interchangeably with protein, peptide and peptide fragments. “Polypeptides” include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. The polypeptides include natural peptides, recombinant peptides, synthetic peptides, or a combination thereof.

Expression vectors are defined herein as DNA sequences that are required for the transcription of cloned copies of genes and the translation of their mRNAs in an appropriate host. Such expression vectors are used to express eukaryotic and prokaryotic genes in plants. Expression vectors include, but are not limited to, cloning vectors, modified cloning vectors, specifically, designed plasmids or viruses. For the purposes of the specification and claims, the terms “expression vector” and “vector” are herein used interchangeably.

According to one embodiment, plant expression vectors are provided containing one or more gene constructs of the presently disclosed subject matter. The plant expression vectors contain the necessary elements to accomplish genetic transformation of plants so that the gene constructs are introduced into the plant's genetic material in a stable manner, i.e., a manner that will allow the genes to be passed on to the plant's progeny. The design and construction of the expression vector influence the integration of the gene constructs into the plant genome and the ability of the genes to be expressed by plant cells.

In one embodiment, the plant expression vector is an Agrobacterium-based expression vector. Various methods are known in the art to accomplish the genetic transformation of plants and plant tissues by the use of Agrobacterium-mediated transformation systems, i.e., A. tumefaciens and A. rhizogenesis. Agrobacterium is the etiologic agent of crown gall, a disease of a wide range of dicotyledons and gymnosperms that results in the formation of tumors or galls in plant tissue at the site of infection. Agrobacterium, which normally infects the plant at wound sites, carries a large extrachromosomal element called Ti (tumor-inducing) plasmid. Ti plasmids contain two regions required for tumor induction. One region is the T-DNA (transferred-DNA) which is the DNA sequence that is ultimately found stably transferred to plant genomic DNA. The other region is the vir (virulence) region which has been implicated in the transfer mechanism. Although the vir region is absolutely required for stable transformation, the vir DNA is not actually transferred to the infected plant. Transformation of plant cells mediated by infection with A. tumefaciens and subsequent transfer of the T-DNA alone have been well documented. See, for example, Bevan et al. (1982), incorporated herein by reference in its entirety.

The presently disclosed subject matter provides transgenic tobacco plants having altered energy compound accumulation in order to optimize the corresponding tobacco biomass for production of bioethanol. In the production of bioethanol from fermentation of extracts of tobacco biomass, the amount of the hexose can be measured as free hexose in the juice of the tobacco biomass, as the total hexose as measured by percent of wet weight of the biomass, or as the total hexose as measured by percent of dry weight of the biomass. As used herein, for the purposes of the specification and claims, the term “tobacco biomass” is intended to broadly encompass the tobacco plants of the present disclosure, including the whole tobacco plant, any tissue or portion of the tobacco plant, juice of the tobacco plant, and the extracted tobacco biomass.

In one or more embodiments, the transgenic tobacco plants having altered energy compound accumulation have elevated accumulation of sugars through expression of corresponding transgenes. For the purposes of the specification and claims, the terms “hexose” and “sugar” are herein used interchangeably. In one or more embodiments, the transgenic tobacco plants having altered sugar accumulation further include conferred tolerance to herbicides through expression of corresponding transgenes. The herbicides include glyphosate and phosphinothricin (PPT)/glufosinate herbicides.

The presently disclosed subject matter provides transgenic tobacco plants having altered energy compound accumulation in order to optimize tobacco biomass for ethanol fermentation. The sucrose-cleaving enzymes of plants have multiple functions that directly or indirectly affect different processes, particularly sugar accumulation in plant storage organs. According to their pH optima, plant invertases can be divided into two categories: neutral and acid invertases. Acid invertases are of exceptional importance as they are the only enzymes able to cleave sucrose in extracellular compartments such as the vacuole (vacuolar invertase; VI) or the apoplastic space (cell wall invertase; CWI). The activity of acid invertases is inhibited by small polypeptides termed Sucrose Invertase Inhibitors (SII's) specific for a particular invertase type. This regulation has been exploited in the presently disclosed subject matter to manipulate carbohydrate metabolism in transgenic plants. Specifically, the expression of two different SII's was directed by regulating the activity of these SII's in specific cell types in transgenic plants to provide higher accumulation of sugars in storage compartments of tobacco plants.

In one aspect, the present disclosure relates to a re-direction of sugar flux between source and sink organs using fine regulation of plant metabolic pathways resulting in higher accumulation of sugars for bioethanol production from tobacco biomass.

In one embodiment of the present disclosure, a genetically modified tobacco plant is provided having altered hexose accumulation, the plant comprising a gene encoding a sucrose invertase inhibitor operably linked to a promoter, such that the hexose accumulation in the modified plant is one or both of increased and altered as compared to a non-genetically modified tobacco plant.

In one embodiment of the genetically modified tobacco plant, the total content of hexose sugar can be increased as compared to a non-genetically modified tobacco plant.

In one embodiment of the present disclosure, a method is provided for producing ethanol from tobacco biomass, the method comprising fermenting a tobacco biomass extract such that ethanol is produced from the fermentation, wherein the tobacco biomass extract is derived from any of the genetically modified tobacco plants having altered hexose accumulation provided herein.

In one embodiment of the genetically modified tobacco plant, the sucrose invertase inhibitor can be an acid sucrose invertase inhibitor. The acid sucrose invertase inhibitor can be an apoplasmic and cell wall sucrose invertase inhibitor protein Nt_INH1. The Nt_INH1 protein can comprise SEQ ID NO: 2. The acid sucrose invertase inhibitor can be a vacuolar sucrose invertase inhibitor protein Nt_INHh. The Nt_INHh protein can comprise SEQ ID NO: 4.

In one embodiment of the genetically modified tobacco plant, the promoter can be a phloem-specific promoter. The phloem-specific promoter can be an Arabidopsis thaliana Sucrose Transporter gene 2 (AtSUC2) promoter and the hexose accumulation in the modified plant can be increased in the phloem as compared to a non-genetically modified tobacco plant. The AtSUC2 promoter can comprise SEQ ID NO: 5.

In one embodiment of the genetically modified tobacco plant, the promoter can be a stem/root-specific promoter. The stem/root-specific promoter can be a rolC promoter and the hexose accumulation in the modified plant can be increased in one or both of the stems and the roots as compared to a non-genetically modified tobacco plant. The rolC promoter can comprise SEQ ID NO: 6.

In one embodiment of the genetically modified tobacco plant, the promoter can be a light-inducible promoter. The light-inducible promoter can be a RuBisCo rbcS promoter. The rbcS promoter can comprise SEQ ID NO: 7.

In one embodiment of the genetically modified tobacco plant, the promoter can be a constitutive promoter. The constitutive promoter can be a CaMV 35S promoter. The CaMV 35S promoter can comprise SEQ ID NO: 8.

In one embodiment of the genetically modified tobacco plant, the plant can further comprise a gene encoding a PAT1 protein operably linked to a promoter, wherein expression of the PAT1 protein confers resistance to a phosphinothricin (PPT) herbicide as compared to a non-genetically modified tobacco plant.

In one embodiment of the genetically modified tobacco plant, the plant can further comprise a gene encoding a enoylpyrovyl-shikimate 3 phosphate synthase (EPSPS) operably linked to a promoter and a chloroplast signal peptide suitable to localize expression of the EPSPS protein to the chloroplast, wherein expression of the chloroplast-localized EPSPS confers resistance to a glyphosate herbicide as compared to a non-genetically modified tobacco plant.

In one embodiment of the present disclosure, a seed is provided of a genetically modified plant having altered hexose accumulation, the plant comprising a gene encoding a sucrose invertase inhibitor operably linked to a promoter, such that the hexose accumulation in the modified plant is one or both of increased and altered as compared to a non-genetically modified tobacco plant.

In one embodiment of the present disclosure, a genetically modified tobacco plant is provided having altered hexose accumulation, the plant comprising a gene encoding an apoplasmic and cell wall sucrose invertase inhibitor protein Nt_INH1 operably linked to an Arabidopsis thaliana Sucrose Transporter gene 2 (AtSUC2) promoter, such that the hexose accumulation in the modified plant is increased in the phloem as compared to a non-genetically modified tobacco plant.

In one embodiment of the present disclosure, a genetically modified tobacco plant is provided having altered hexose accumulation, the plant comprising a gene encoding a vacuolar sucrose invertase inhibitor protein Nt_INHh operably linked to an Arabidopsis thaliana Sucrose Transporter gene 2 (AtSUC2) promoter, such that the hexose accumulation in the modified plant is increased in the phloem as compared to a non-genetically modified tobacco plant.

In one embodiment of the present disclosure, a genetically modified tobacco plant is provided having altered hexose accumulation, the plant comprising a gene encoding an apoplasmic and cell wall sucrose invertase inhibitor protein Nt_INH1 operably linked to a rolC promoter, such that the hexose accumulation in the modified plant is increased in one or both of the stems and the roots as compared to a non-genetically modified tobacco plant.

In one embodiment of the present disclosure, a genetically modified tobacco plant is provided having altered hexose accumulation, the plant comprising a gene encoding a vacuolar sucrose invertase inhibitor protein Nt_INHh operably linked to a rolC promoter, such that the hexose accumulation in the modified plant is increased in one or both of the stems and the roots as compared to a non-genetically modified tobacco plant.

In one embodiment of the present disclosure, a genetically modified tobacco plant is provided having altered hexose accumulation, the plant comprising a gene encoding an apoplasmic and cell wall sucrose invertase inhibitor protein Nt_INH1 operably linked to a light-inducible rbcS promoter, such that the hexose accumulation in the modified plant is one or both of increased and altered as compared to a non-genetically modified tobacco plant.

In one embodiment of the present disclosure, a genetically modified tobacco plant is provided having altered hexose accumulation, the plant comprising a gene encoding a vacuolar sucrose invertase inhibitor protein Nt_INHh operably linked to a light-inducible rbcS promoter, such that the hexose accumulation in the modified plant is one or both of increased and altered as compared to a non-genetically modified tobacco plant.

In one embodiment of the present disclosure, a genetically modified tobacco plant is provided having altered hexose accumulation, the plant comprising a gene encoding an apoplasmic and cell wall sucrose invertase inhibitor protein Nt_INH1 operably linked to a constitutive CaMV 35S promoter, such that the hexose accumulation in the modified plant is one or both of increased and altered as compared to a non-genetically modified tobacco plant.

In one embodiment of the present disclosure, a genetically modified tobacco plant is provided having altered hexose accumulation, the plant comprising a gene encoding a vacuolar sucrose invertase inhibitor protein Nt_INHh operably linked to a constitutive CaMV 35S promoter, such that the hexose accumulation in the modified plant is one or both of increased and altered as compared to a non-genetically modified tobacco plant.

In one embodiment of the present disclosure, the genetically modified tobacco plant having altered hexose accumulation can further comprise a gene encoding a PAT1 protein operably linked to a promoter, wherein expression of the PAT1 protein confers resistance to a phosphinothricin (PPT) herbicide as compared to a non-genetically modified tobacco plant.

In one embodiment of the present disclosure, the genetically modified tobacco plant having altered hexose accumulation can further comprise a gene encoding a enoylpyrovyl-shikimate 3 phosphate synthase (EPSPS) operably linked to a promoter and a chloroplast signal peptide suitable to localize expression of the EPSPS protein to the chloroplast, wherein expression of the chloroplast-localized EPSPS confers resistance to a glyphosate herbicide as compared to a non-genetically modified tobacco plant.

In one embodiment of the present disclosure, a vector is provided for transformation of a tobacco plant, the vector comprising a gene encoding a sucrose invertase inhibitor operably linked to a promoter, such that expression of the sucrose invertase inhibitor in the tobacco plant one of alters and increases hexose accumulation in the plant. In the vector for transformation of a tobacco plant, the expression of the sucrose invertase inhibitor in the tobacco plant can increase hexose accumulation in the plant.

In one embodiment of the present disclosure, a method is provided for producing ethanol from tobacco biomass, the method comprising fermenting a tobacco biomass extract wherein ethanol is produced from the fermentation, wherein the tobacco biomass extract is derived from a tobacco plant that has one of increased and altered hexose accumulation as a result of having been transformed with one of the expression vectors provided herein.

In one embodiment of the vector for transformation of a tobacco plant, the sucrose invertase inhibitor can be an acid sucrose invertase inhibitor. The acid sucrose invertase inhibitor can be an apoplasmic and cell wall sucrose invertase inhibitor protein Nt_INH1 comprising SEQ ID NO: 2. The acid sucrose invertase inhibitor can be a vacuolar sucrose invertase inhibitor protein Nt_INHh comprising SEQ ID NO: 4.

In one embodiment of the vector for transformation of a tobacco plant, the promoter can be a phloem-specific promoter and hexose accumulation can be increased in the phloem of the tobacco plant. The phloem-specific promoter can be an Arabidopsis thaliana Sucrose Transporter gene 2 (AtSUC2) promoter comprising SEQ ID NO: 5.

In one embodiment of the vector for transformation of a tobacco plant, the promoter can be a stem/root-specific promoter and hexose accumulation can be increased in one or both of the stems and the roots of the tobacco plant. The stem/root-specific promoter can be a rolC promoter comprising SEQ ID NO: 6.

In one embodiment of the vector for transformation of a tobacco plant, the promoter can be a light-inducible promoter. The light-inducible promoter can be a RuBisCo rbcS promoter comprising SEQ ID NO: 7.

In one embodiment of the vector for transformation of a tobacco plant, the promoter can be a constitutive promoter. The constitutive promoter can be a CaMV 35S promoter comprising SEQ ID NO: 8.

In one embodiment of the present disclosure, a vector is provided for transformation of a tobacco plant, the vector comprising a gene encoding an apoplasmic and cell wall sucrose invertase inhibitor protein Nt_INH1 operably linked to an Arabidopsis thaliana Sucrose Transporter gene 2 (AtSUC2) promoter, such that expression of the Nt_INH1 protein in the tobacco plant increases hexose accumulation in the phloem of the tobacco plant.

In one embodiment of the present disclosure, a vector is provided for transformation of a tobacco plant, the vector comprising a gene encoding a vacuolar sucrose invertase inhibitor protein Nt_INHh operably linked to an Arabidopsis thaliana Sucrose Transporter gene 2 (AtSUC2) promoter, such that expression of the Nt_INHh protein in the tobacco plant increases hexose accumulation in the phloem of the tobacco plant.

In one embodiment of the present disclosure, a vector is provided for transformation of a tobacco plant, the vector comprising a gene encoding an apoplasmic and cell wall sucrose invertase inhibitor protein Nt_INH1 operably linked to a stem/root-specific rolC promoter, such that expression of the Nt_INH1 protein in the tobacco plant increases hexose accumulation in one or both of the stems and the roots of the tobacco plant.

In one embodiment of the present disclosure, a vector is provided for transformation of a tobacco plant, the vector comprising a gene encoding a vacuolar sucrose invertase inhibitor protein Nt_INHh operably linked to a stem/root-specific rolC promoter, such that expression of the Nt_INHh protein in the tobacco plant increases hexose accumulation in one or both of the stems and the roots of the tobacco plant.

In one embodiment of the present disclosure, a vector is provided for transformation of a tobacco plant, the vector comprising a gene encoding an apoplasmic and cell wall sucrose invertase inhibitor protein Nt_INH1 operably linked to a light-inducible RuBisCo rbcS promoter, such that expression of the Nt_INH1 protein in the tobacco plant one or both of increases and alters hexose accumulation in the tobacco plant.

In one embodiment of the present disclosure, a vector is provided for transformation of a tobacco plant, the vector comprising a gene encoding a vacuolar sucrose invertase inhibitor protein Nt_INHh operably linked to a light-inducible RuBisCo rbcS promoter, such that expression of the Nt_INHh protein in the tobacco plant one or both of increases and alters hexose accumulation in the tobacco plant.

In one embodiment of the present disclosure, a vector is provided for transformation of a tobacco plant, the vector comprising a gene encoding an apoplasmic and cell wall sucrose invertase inhibitor protein Nt_INH1 operably linked to a constitutive CaMV 35S promoter, such that expression of the Nt_INH1 protein in the tobacco plant one or both of increases and alters hexose accumulation in the tobacco plant.

In one embodiment of the present disclosure, a vector is provided for transformation of a tobacco plant, the vector comprising a gene encoding a vacuolar sucrose invertase inhibitor protein Nt_INHh operably linked to a constitutive CaMV 35S promoter, such that expression of the Nt_INHh protein in the tobacco plant one or both of increases and alters hexose accumulation in the tobacco plant.

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### EXAMPLES

### Example 1

### Generation of Transgenic Tobacco Plants Expressing Sucrose Invertase Inhibitors under Control of a Phloem-Specific Promoter

Construction of the Expression Cassette. Tobacco gene Nt-inh1 (GenBank Accession No: Y12805.1; SEQ ID NO: 1) encoding Apoplasmic and Cell Wall Sucrose Invertase Inhibitor protein (SEQ ID NO: 2) was optimized for cloning in plant binary vectors of pBIN19 series (Bevan M., 1984) for the expression in transformed tobacco cells and synthesized using GENEART resulting in pMAT-Nt_inh1 plasmid DNA. Similarly, tobacco gene Nt-inhh (GenBank Accession No: Y12806.1; SEQ ID NO: 3) encoding Vacuolar Sucrose Invertase Inhibitor protein (SEQ ID NO: 4) was optimized for cloning in plant binary vectors of pBIN19 series for the expression in transformed tobacco cells and synthesized using GENEART resulting in pMAT-Nt_inhh plasmid DNA.

To target expression of the sucrose invertase inhibitor genes in the sink/storage parts of tobacco plants, a binary vector was designed using the backbone of the widely used Agrobacterium binary vector for plant transformation pBIN19. Specifically, this was achieved by substitution of the CaMV 35S promoter in the original pBIN19 vector with the phloem-specific promoter from Arabidopsis thaliana, Sucrose Transporter gene 2 (AtSUC2; GenBank: JQ733913.1; SEQ ID NO: 5). A DNA fragment containing the AtSUC2 promoter was synthesized using GENEART and designed for cloning into Hind III-Xba I restriction site resulting in a plasmid pMK-RQ_At_SUC2.

Vector pBIN19 was digested with Hind III-Xba I and ligated with the corresponding Hind III-Xba I AtSUC2 promoter fragment. Kanamycin resistant clones were selected and screened on agarose gel after treatment with corresponding enzymes resulting in the vector pBI_SUC2. Xba I-Sac I fragments encoding the Nt_inh1 gene from the pMAT-Nt_inh1 plasmid and the Nt_inhh gene from the pMAT-Nt_inhh plasmid were then cloned into pBI_SUC2 binary vector resulting in pBI_SUC2-Nt_inh1 vector and pBI_SUC2-Nt_inhh vector, respectively. Finally, the resulting vectors were transformed by electroporation into Agrobacterium tumefaciens LBA4404 cells for further transformation of plants.

Stable Transformation of Tobacco Plants. Stable tobacco plant transformation was performed as described in Marc De Block et al., 1989, which is incorporated herein by reference in its entirety. Specifically, tobacco leaf discs of different Nicotiana tabacum varieties SHIREY LC, TND 950, K 326, and NC 567 (all varieties were from F.W. RICKARD SEEDS, INC) were transformed with the kanamycin-resistant pBI_SUC2-Nt_inh1 and pBI_SUC2-Nt_inhh vector constructs. Various transgenic plants were identified as described in Randhawa et al., 2009. Various T2 transgenic lines (S76; SHIREY LC pBI_SUC2-Nt_inh1 line #6; S83; SHIREY LC pBI_SUC2-Nt_inhh line #3; TN81; TND950 pBI_SUC2-Nt_inhh line # 1; TN85 pBI_SUC2-Nt_inhh line #5; NC85 pBI_SUC2-Nt_inhh line #5; NC87 pBI_SUC2-Nt_inhh line #7; K811 pBI_SUC2-Nt_inhh line #11; K813 pBI_SUC2-Nt_inhh line #13; and S79 pBI_SUC2-Nt_inh1 line 9 were selected and tested by genomic DNA PCR for the presence of selectable marker NPT-II transcripts.

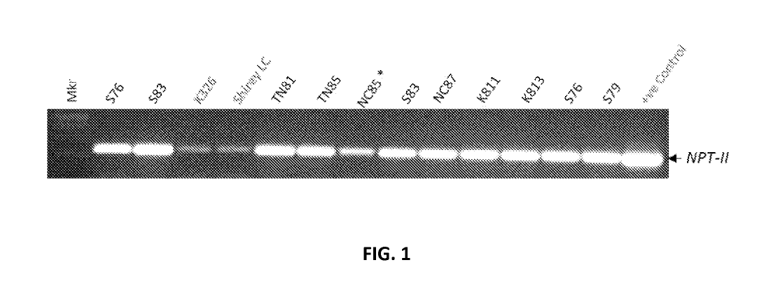

Expression Profiling. Expression profiling of the transgenic tobacco having the pBI_SUC2-Nt_inh1 and pBI_SUC2-Nt_inhh vector constructs was performed as follows. RNA was extracted from floral organ tissues of the transgenic tobacco plants and cDNA was prepared for all the identified transgenic plants. Primer pairs specific to the selectable marker gene neomycin phosphotransferase II (NPT-II) associated with the transgene (inh1 or inhh) in the T-DNA were used to amplify the NPT-II gene. Untransformed tobacco plants (lines: K326 and SHIREY LC) served as negative control in the experiment and either one of the plasmid constructs (pBI_SUC2-Nt_inh1 or pBI_SUC2-Nt_inhh) containing the NPT-II gene served as the positive control. The results showed that strong amplification was observed only in the samples from the transgenic tobacco plants as compared to untransformed tobacco plants (see FIG. 1).

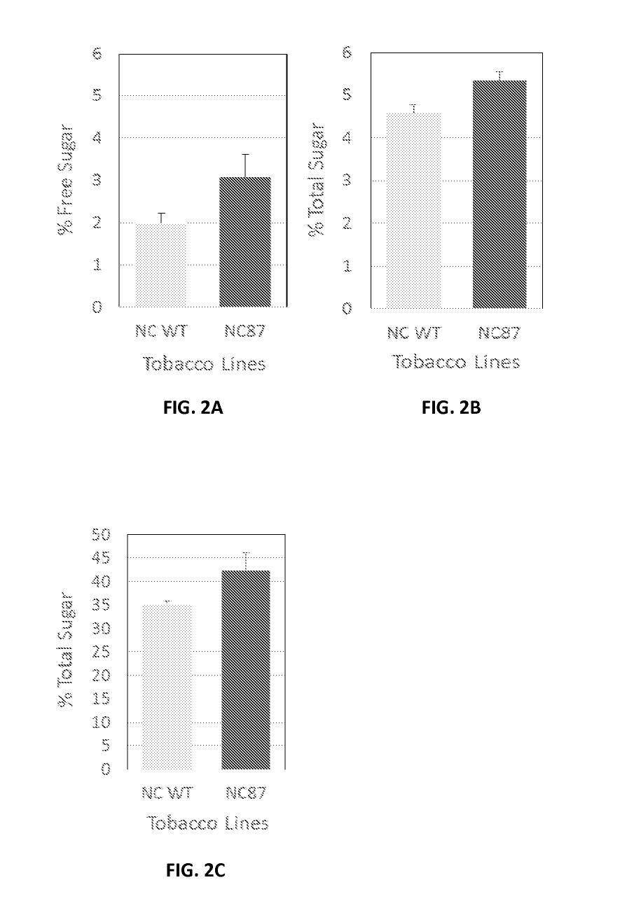

Analysis of Sugar Content in Transgenic Tobacco Tissue. The amount of sugar present in the transgenic tobacco lines was quantified and compared to the amount of sugar in untransformed control plants. Specifically, free hexose from the juice of the tobacco plants and total hexose from the tobacco plant biomass was analyzed. The experiment was performed with the transgenic tobacco line NC 87, which is a NC 567 tobacco variety containing the pBI_SUC2-Nt_inhh vector construct. The untransformed NC 567 tobacco variety was used as the wild-type control and referred to as “NC WT”. The plants were grown in soil under greenhouse conditions. In this experiment, the juice was extracted from 3 month old plants using a woodchip shredder followed by grinding of the tobacco in a blender and squeezing out the juice. The percent hexose in this tobacco juice was calculated. The remaining tobacco biomass was separated and analyzed for total hexose analysis in both wet and dry weight estimates. Analysis of the hexose in both the tobacco juice and biomass was performed using standard procedures that included phenol/sulphuric acid assay described in Kimberley A.C.C. Taylor, 1995. The results of this analysis are shown in FIGS. 2A-2C.

Specifically, FIGS. 2A-2C are histograms showing the hexose levels in the untransformed NC WT tobacco compared to levels in the transgenic tobacco line NC 87 as follows: A) Histogram showing the percent free hexose measured in the juice (Free Sugar) for each of the transgenic (NC 87) and wild-type (NC WT) lines; B) Histogram showing the total hexose as measured by percent of wet weight of the biomass (Total Sugar) for each of the transgenic (NC 87) and wild-type (NC WT) lines; and C) Histogram showing the total hexose as measured by percent of dry weight of the biomass (Total Sugar) for each of the transgenic (NC 87) and wild-type (NC WT) lines. The data in FIG. 2A shows that free hexose (sugar) in the tobacco juice was increased by about 50% in the transgenic line as compared to the untransformed wild-type variety (NC WT). Total hexose sugar in the biomass of the transgenic tobacco was also increased by about 9% relative to the wild-type tobacco biomass (FIG. 2B). The increase in total hexose sugar in the biomass of the transgenic tobacco over the wild-type control was calculated to be about 5% on a dry weight basis (FIG. 2C).

In the context of developing tobacco plants for enhanced bioethanol production, such a large increase in total hexose sugar in the tobacco plant juice shown in FIG. 2A is definitely desirable. For example, this increase in hexose in the juice can allow for substantial improvements in both quantity and efficiency in recovery of the hexose sugar from the plant. In addition, the overall increase in the total hexose sugar found in the biomass of the transgenic tobacco is similarly favorable for enhanced bioethanol production (FIGS. 2B-2C). Tobacco modified according to this presently disclosed subject matter has the potential to substantially increase the amount of ethanol produced per acre than non-modified tobacco. Further, because tobacco is a non-food plant that can thrive in poor soil, it does not compete with food-producing plants such as corn and soybeans for more fertile soil.

### Example 2

### Generation of Transgenic Tobacco Plants Expressing Sucrose Invertase Inhibitors under Control of a Root/Stem-Specific Promoter

Construction of the Expression Cassette. To target expression of the sucrose invertase inhibitor genes in sink/storage parts of tobacco plants, a binary vector was designed using the backbone of the widely used Agrobacterium binary vector for plant transformation pBIN19. Specifically, this was achieved by substitution of the CaMV 35S promoter in the original pBIN19 vector with the root-stem specific promoter from Agrobacterium rhisogenes rolC (GenBank: JQ733911.1; SEQ ID NO: 6). A DNA fragment containing the rolC promoter was synthesized using GENEART designed for cloning into Hind III-Xba I restriction site resulting in a plasmid pMA-rolC.

Vector pBIN19 was digested with Hind III-Xba I and ligated with the corresponding Hind III-Xba I rolC promoter fragment. Kanamycin resistant clones were selected and screened on agarose gel after treatment with corresponding enzymes resulting in the vector pBI_rolC. Xba I-Sac I fragments encoding the Nt_inh1 gene from the pMAT-Nt_inh1 plasmid and the Nt_inhh gene from the pMAT-Nt_inhh plasmid were then cloned into the pBI_rolC binary vector resulting in pBI_rolC-Nt_inh1 and pBI_rolC-Nt_inhh vectors, respectively. Finally, the resulting vectors were transformed by electroporation into Agrobacterium tumefaciens LBA4404 cells for further transformation of plants.

Stable Transformation of Tobacco Plants. Stable plant transformation was performed as described above in Example 1 except that the pBI_rolC-Nt_inh1 and pBI_rolC-Nt_inhh vectors were used rather than the pBI_SUC2-Nt_inh1 and pBI_SUC2-Nt_inhh vectors.

### Example 3

### Generation of Transgenic Tobacco Plants Ectopically Expressing Sucrose Invertase Inhibitors under RuBisCo rbcS Promoter

Construction of the Expression Cassette. The Nt-inh1 and Nt-inhh nucleotide sequences described in Example 1 were sub-cloned into pBI19 based binary vector using Xba I and Sac I cloning sites under regulation of Asteraceous chrysanthemum rbcS promoter (SEQ ID NO: 7; Outchkourov et al., 2003) resulting in plant expression vectors pBI_rbcS_Nt-inh1 and pBI_rbcS_Nt-inhh, respectively. The pBI_rbcS_Nt-inh1 and pBI_rbcS_Nt-inhh vectors were transformed by electroporation into A. tumefaciens LBA4404 cells for further transformation of plants.

Stable Transformation of Tobacco Plants. Stable plant transformation is performed as described above in Example 1 except that the pBI_rbcS_Nt-inh1 and pBI_rbcS_Nt-inhh vectors are used rather than the pBI_SUC2-Nt_inh1 and pBI_SUC2-Nt_inhh vectors.

### Example 4

### Generation of Transgenic Tobacco Plants Ectopically Expressing Sucrose Invertase Inhibitors under a Constitutive Promoter

Construction of the Expression Cassette. The Nt-inh1 and Nt-inhh nucleotide sequences described in Example 1 were sub-cloned into pBI19 based conventional pBI121 binary vectors (CLONTECH) using Xba I and Sac I cloning sites under regulation of the constitutive CaMV 35S promoter (SEQ ID NO: 8). The resulting vectors pBI_35S_Nt-inh1 and pBI_35S_Nt-inhh were transformed by electroporation into Agrobacterium tumefaciens LBA4404 cells for further transformation of plants.

Stable Transformation of Tobacco Plants. Stable plant transformation is performed as described above in Example 1 except that the pBI_35S_Nt-inh1 and pBI_35S_Nt-inhh vectors are used rather than the pBI_SUC2-Nt_inh1 and pBI_SUC2-Nt_inhh vectors.

### Example 5

### Generation of Transgenic Tobacco Plants having Altered Hexose Expression and Expressing PPT (BASTA) Resistance Gene

Construction of the Expression Cassette. Resistance to phosphinothricin (PPT) was conferred to the transgenic constructs described above in Examples 1-4 according to the following procedures. An artificial phosphinothricin (PPT) resistance gene (GenBank Accession No. A02774; SEQ ID NO: 9), encoding a PAT1 protein, was used for construction of PPT tolerance. The PPT gene was PCR-amplified from the original p35SAc plasmid (USDA Collection) with the following primers: AscI-PAT.F: 5′-gCT Tgg CgC gCC CAT ggA gTC AAA gAT TCA-3′ (SEQ ID NO: 10) and NheI-PAT.R: 5′-gCT TgC TAg CgA gCT Cgg TAC CCA CTg gA-3′ (SEQ ID NO: 11) and cloned into the AscI-NheI restriction site of the previously generated pBI_vectors described in Examples 1-4 above such that the Kanamycin plant selection marker was replaced with the PPT resistance gene. Kamamycin resistant clones were selected and screened on agarose gel after treatment with corresponding enzymes to generate the constructs for plant transformation. In this manner the PPT gene was placed under the control of pBI-derived NOS promoter and terminator. Each of the vector DNA structures was verified with PCR and restriction enzyme digest analysis. Finally, the resulting vectors were transformed by electroporation into Agrobacterium tumefaciens LBA4404 cells for transformation of plants.

Stable Transformation of Tobacco Plants. Stable tobacco plant transformation was performed as described in Marc De Block et al., 1989, which is incorporated herein by reference in its entirety. Specifically, tobacco leaf explants of the variety Nicotiana tabacum cv. Wisconsin 38, SHIREY LC, TND 950, K 326, and NC 567 were transformed with the vector constructs described above. Transgenic lines were selected on glufosinate (5 mg/L) (BASTA; CRESCENT CHEMICAL, Islandia, N.Y.) and tested by PCR and/or Western blotting for the presence of the PAT1 gene. The best transgenic tobacco lines with altered hexose accumulation due to expression of the sucrose invertase inhibitor genes were selected among the primary transformants.

Transgenic tobacco lines are later maintained in soil, and subsequent generations (T1 and T2) are obtained by self-fertilization. The transgenic tobacco lines are further tested for tolerance to glufosinate by spraying 1-2 week old plants grown in a greenhouse with 5 mg/L glufosinate.

### Example 6

### Generation of Transgenic Tobacco Plants having Altered Hexose Expression and Expressing Glyphosate Resistance Gene

Construction of the Expression Cassette. Resistance to glyphosate was conferred to the transgenic constructs described above in Examples 1-4 according to the following procedures. A gene encoding a mutant enoylpyrovyl-shikimate 3 phosphate synthase (EPSPS) (GenBank Accession No: EU477376.1; SEQ ID NO: 12) with enhanced tolerance to the herbicide glyphosate was used to generate transgenic tobacco plants having altered hexose expression and a tolerance to glyphosate. A chloroplast signal peptide derived from Brassica napus cv. Wistar ribulose-1,5-bisphosphate carboxylase/oxygenase small subunit (SEQ ID NO: 13) was used for chloroplast localization of the expressed EPSPS enzyme. The whole synthetic glyphosate resistance gene (GLY^{R} gene) including the nucleotide sequence encoding the mutant EPSPS and the nucleotide sequence encoding the chloroplast signal sequence (SEQ ID NO: 14) was optimized for the expression in transformed tobacco cells and for cloning. Nhe I-Asc I restriction enzymes were used to clone into the Asc I-Nhe I restriction site of the previously generated pBI_{—} vectors from Examples 1-4 in order to substitute the kanamycin resistance gene with the glyphosate resistance gene (GLY^{R} gene). The GLY^{R} gene was synthesized by GENEART. Vectors pBI from Examples 1-4 were digested with Nhe I-Asc I and ligated with the corresponding Nhe I-Asc I GLY^{R} gene fragment. Kanamycin resistant clones were selected and screened on agarose gel after treatment with corresponding enzymes. Each of the vector DNA structures was verified with PCR and restriction enzyme digest analysis. In this manner the glyphosate resistance gene was placed under the control of pBI-derived NOS promoter and terminator.

Stable Transformation of Tobacco Plants. The resulting vectors were transformed by electroporation into Agrobacterium tumefaciens LBA4404 cells for transformation of plants. Stable plant transformation was performed as described above in Example 5 except that selection of transgenic lines was performed with glyphosate. The best transgenic tobacco lines with altered hexose accumulation due to expression of the sucrose invertase inhibitor genes were selected among the primary transformants.

Transgenic tobacco lines are later maintained in soil, and subsequent generations (T1 and T2) are obtained by self-fertilization. The transgenic tobacco lines are further tested for tolerance to glyphosate by spraying 1-2 week old plants grown in a greenhouse with 0.1 mM solution of glyphosate.

### REFERENCES

Andrianov V, et al. Tobacco as a production platform for biofuel: overexpression of Arabidopsis DGAT and LEC2 genes increases accumulation and shifts the composition of lipids in green biomass. Plant Biotechn. J., 8:277-287 (2010).

Greiner S, Rausch Th, Sonnewald U, and Herbers K. Ectopic expression of a tobacco invertase inhibitor homolog prevents cold-induced sweetening of potato tubers. Nature Biotechnology, 17:708-711 (1999).

Outchkourov N S, Peters J, Jong J, Rademakers W, Jongsma M A. The promoter-terminator of chrysanthemum rbcS 1 directs very high expression levels in plants. Planta 216:1003-1012 (2003).

Marc De Block, Dirk De Brouwer, Paul Tenning. Transformation of Brassica napus and Brassica oleracea using Agrobacterium tumefaciens and the expression of the bar and neo genes in the transgenic plants. Plant Physiol. 91:694-701 (1989).

Bevan, M. et al, Int. Rev. Genet. 16:357 (1982).

Bevan, M. Binary Agrobacterium vectors for plant transformation. Nucleic Acids Res. 12:8711-8721(1984).

Randhawa et al. Multiplex PCR-Based Simultaneous Amplification of Selectable Marker and Reporter Genes for the Screening of Genetically Modified Crops. J. Agric. Food Chem. 57(12):5167-5172 (2009).

Kimberley A. C. C. Taylor. A modification of the phenol/sulfuric acid assay for total carbohydrates giving more comparable absorbances. Applied Biochemistry and Biotechnology. 53(3):207 (1995).

Any patents or publications mentioned in this specification are indicative of the levels of those skilled in the art to which the present disclosure pertains. These patents and publications are herein incorporated by reference in their entirety to the same extent as if each individual publication was specifically and individually indicated to be incorporated by reference.

One skilled in the art will readily appreciate that the present disclosure is well adapted to carry out the objects and obtain the ends and advantages mentioned, as well as those inherent therein. The present Examples along with the methods described herein are presently representative of preferred embodiments, are exemplary, and are not intended as limitations on the scope of the invention. Changes therein and other uses will occur to those skilled in the art which are encompassed within the spirit of the present disclosure as defined by the scope of the claims.

1. A genetically modified tobacco plant having altered hexose accumulation, the plant comprising a gene encoding a sucrose invertase inhibitor operably linked to a phloem-specific promoter or a stem/root-specific promoter, wherein hexose accumulation in the modified plant is one or both of increased or altered as compared to a non-genetically modified tobacco plant.

2. The genetically modified plant of claim 1, wherein the sucrose invertase inhibitor is an acid sucrose invertase inhibitor.

3. The genetically modified plant of claim 2, wherein the acid sucrose invertase inhibitor is an apoplasmic and cell wall sucrose invertase inhibitor protein Nt_INH1.

4. The genetically modified plant of claim 3, wherein the Nt_INH1 protein comprises SEQ ID NO: 2.

5. The genetically modified plant of claim 2, wherein the acid sucrose invertase inhibitor is a vacuolar sucrose invertase inhibitor protein Nt_INHh.

6. The genetically modified plant of claim 5, wherein the Nt_INHh protein comprises SEQ ID NO: 4.

7. (canceled)

8. The genetically modified plant of claim 1, wherein the phloem-specific promoter is an Arabidopsis thaliana Sucrose Transporter gene 2 (AtSUC2) promoter and the hexose accumulation in the modified plant is increased in the phloem as compared to a non-genetically modified tobacco plant.

9. The genetically modified plant of claim 8, wherein the AtSUC2 promoter comprises SEQ ID NO: 5.

10. (canceled)

11. The genetically modified plant of claim 1, wherein the stem/root-specific promoter is a roIC promoter and the hexose accumulation in the modified plant is increased in one or both of the stems and the roots as compared to a non-genetically modified tobacco plant.

12. The genetically modified plant of claim 11, wherein the roIC promoter comprises SEQ ID NO: 6.

13-18. (canceled)

19. The genetically modified plant of claim 1, further comprising a sequence encoding a PAT1 protein, wherein expression of the PAT1 protein confers resistance to a phosphinothricin (PPT) herbicide as compared to a non-genetically modified tobacco plant.

20. The genetically modified plant of claim 1, further comprising a sequence encoding a enoylpyrovyl-shikimate 3 phosphate synthase (EPSPS) operably linked to a promoter and a chloroplast signal peptide suitable to localize expression of the EPSPS protein to the chloroplast, wherein expression of the chloroplast-localized EPSPS confers resistance to a glyphosate herbicide as compared to a non-genetically modified tobacco plant.

21. A seed of the plant of claim 1.

22-31. (canceled)

32. An expression vector for transformation of a tobacco plant, the vector comprising a gene encoding a sucrose invertase inhibitor operably linked to a phloem-specific promoter or a stem/root-specific promoter, wherein expression of the sucrose invertase inhibitor in the tobacco plant one of increases or alters hexose accumulation in the plant.

33. The expression vector of claim 32, wherein the sucrose invertase inhibitor is an acid sucrose invertase inhibitor.

34. The expression vector of claim 33, wherein the acid sucrose invertase inhibitor is an apoplasmic and cell wall sucrose invertase inhibitor protein Nt_INH1 comprising SEQ ID NO: 2.

35. The expression vector of claim 33, wherein the acid sucrose invertase inhibitor is a vacuolar sucrose invertase inhibitor protein Nt_INHh comprising SEQ ID NO: 4.

36. (canceled)

37. The expression vector of claim 32, wherein the phloem-specific promoter is an Arabidopsis thaliana Sucrose Transporter gene 2 (AtSUC2) promoter comprising SEQ ID NO: 5.

38. (canceled)

39. The expression vector of claim 32, wherein the stem/root-specific promoter is a roIC promoter comprising SEQ ID NO: 6.

40-51. (canceled)

52. A method for producing ethanol from tobacco biomass, the method comprising fermenting a tobacco biomass extract such that ethanol is produced from the fermentation, wherein the tobacco biomass extract is derived from the genetically modified tobacco plant of claim 1.

53. (canceled)
